(19) United States
(12) Patent Application Publication
(10) Pub. No.: US 2014/0276905 A1
(43) Pub. Date: Sep. 18, 2014

(54) Insertion Element and Insertion Device

(71) Applicant: BIOTRONIK AG, Bulach (CH)

(72) Inventors: Louis Schaer, Bern (CH); Amir Fargahi, Buelach (CH)

(73) Assignee: BIOTRONIK AG, Bulach (CH)

(21) Appl. No.: 14/197,065

(22) Filed: Mar. 4, 2014

Related U.S. Application Data
(60) Provisional application No. 61/802,759, filed on Mar. 18, 2013.

(51) Int. Cl. A61F 2/954; A61M 25/00

(52) U.S. Cl. CPC A61F 2/954; A61M 25/0045; A61M 25/0074
USPC 606/108

(57) ABSTRACT

An insertion element (100), which is designed to cooperate with an insertion device (200) for inserting a medical implant (222) into a human or animal body. The insertion element (100) comprises a sleeve (10) with a distal end (40) and a proximal end (30) opposite the distal end (40), said sleeve also having at least one expandable region (10a), which can be transferred from a first state of small clear cross section at least at the proximal end (30) into a second state of larger clear cross section at least at the proximal end (30), wherein the expandable region (10a) of the sleeve (10) is flatter in the first state than in the second state.

### CROSS REFERENCE TO RELATED APPLICATIONS

This application claims benefit of priority to U.S. patent application Ser. No. 61/802,759 filed Mar. 18, 2013; the entire content of which is herein incorporated by reference in its entirety.

### TECHNICAL FIELD

The invention relates to an insertion element and also to an insertion device for inserting a medical implant into a human or animal body.

### BACKGROUND

In the field of medicine, implants are often used that are introduced into an animal and/or human body either permanently or at least for a relatively long period of time in order to carry out replacement functions. For example, these implants could include heart pacemakers, brain pacemakers for Parkinson's patients, cardiac implants, cochlear implants, retinal implants, dental implants, implants for joint replacement, vessel prostheses, or stents.

Before insertion into the body, implants are connected to catheters, with the aid of which they can be placed precisely and released in a defined manner at the site of use. For introduction into the animal and/or human body, a tubular insertion element is used, through which the implant is slid by means of the insertion device. In order to avoid the stressing of the vessel during introduction of the implant, an insertion element is known from US 2010/0094392 A1, which is only expanded to the necessary larger diameter once the implant has been passed through The insertion element consists of two to three coaxially arranged layers, wherein the outer layer is provided with longitudinal slits, which enables the enlargement of the diameter.

Conventional insertion elements are often too large however for use in small animals.

One object of the invention is to specify an insertion element that enables insertion of an implant even in areas having small dimensions.

A further object can be considered that of providing a corresponding insertion device.

### SUMMARY

An insertion element is provided, which is designed to cooperate with an insertion device to for inserting a medical implant into a human or animal body. The insertion element comprises a sleeve with a distal end and a proximal end opposite the distal end, the sleeve also having at least one expandable region, which can be transferred from a first state of small clear cross section at least at the proximal end into a second state of larger clear cross section at least at the proximal end, wherein the expandable region of the sleeve is flatter in the first state than in the second state.

When the insertion element is handled as intended, the proximal end faces a user and the distal end faces the implant. Due to the ductility of the insertion element when leading the implant or a catheter containing the implant through, the opening in the body for insertion of the implant can be smaller than if a sheath having a maximally required diameter were used.

The insertion element has a small, quasi two-dimensional profile in the first state, at least in its expandable region, and can therefore also be guided through very small cuts or openings. The insertion element can be easily transferred from the first state into the expanded, second state and thus used as a sheath for a multiplicity of diameters of insertion devices, such as catheters, for inserting an implant into the human or animal body. For example, the insertion element can be used for stent implantation in small animals, for example mice.

An insertion element with a quasi two-dimensional profile is understood within the scope of this application to mean that the insertion element has a plane of maximum extension in the first state, at least in its expandable region. Along any axis perpendicular to this plane, the extension is at most 0.5 times the maximum extension, preferably at most 0.25 times the maximum extension, more preferably at most 0.1 times the maximum extension.

In accordance with a favorable embodiment, the expandable region of the sleeve of the insertion element can be unfolded, and/or opened and/or fanned out from the first state into the second state. The design of the insertion element that can be easily unfolded/opened/fanned out enables the compatibility of the insertion element with many different catheter diameters.

In accordance with a favorable embodiment, the sleeve or the expandable region of the sleeve may have an arrowhead-like contour in the first state, for example in the form of a triangle. Other profiles are also conceivable.

In accordance with a favorable embodiment, the sleeve of the insertion element may have a long, drawn-out tip at the distal end. The tip allows improved placement of the insertion element and also of the insertion device, for example in a narrow vessel. The tip can be attached to the expandable region, or the expandable region may transition into the tip. Here, it may be that the tip is not expanded when the expandable region of the sleeve is expanded with the transition from the first state into the second state of the sleeve. The tip may have a length that accounts for a significant proportion of the length of the expandable region up to a multiple of the longitudinal extension of the expandable region. For example, the length of the tip may be half as long as the expandable region.

In accordance with a favorable variant, the sleeve may have a seam, wherein, when opening the seam, the interior of the expandable region can be exposed. The seam may be a breaking seam, for example a seam with a perforation that can be easily broken. The seam allows simple removal of the insertion element after insertion of the insertion device, for example a catheter.

In an advantageous embodiment, the sleeve may have a wall thickness in the insertable region, which is provided as a sheath for the medical implant, of at most 100 μm, preferably at most 70 μm, and more preferably at most 50 μm. A particularly low height of the expandable region of the sleeve is thus possible if the sleeve is in the first, unexpanded, flat state. Since the profile in the unexpanded state is quasi “two-dimensional”, the insertion element can be easily inserted into a small cut.

In a favorable embodiment, the sleeve may be formed, at least in some regions, from a metal film having a wall thickness of at most 100 μm, preferably at most 70 μm, and more preferably at most 50 μm. A metal film is mechanically very stable and, for example, can be folded and unfolded particularly easily. It is also conceivable for the sleeve to be formed, at least in some regions, from a polymer film having a wall thickness of at most 100 μm, preferably at most 70 μm, and more preferably at most 50 μm.

The entire sleeve may advantageously have the thin wall thickness, whereby a one-piece insertion element can be easily produced from an appropriate film. Suitable polymer materials are polyether ether ketone (PEEK), polyimide (PI), polyether ketone ketone (PEKK), is polyamides, polyester, polyethylene (PE), polypropylene (PP), and polycarbonate (PC). For example, polyamides such as polyamide 12 (PA-12, consisting of laurin lactam or ω-aminododecanoic acid), or polyesters such as polyethylene terephthalate, known as PET, are preferred. A suitable metal for example is titanium, surgical steel, or the like.

In an advantageous embodiment, the sleeve may have a friction-reducing coating. This can be provided on the inner side, which facilitates the insertion of the insertion device, on the outer side, which facilitates the insertion of the insertion element into the body, and on both sides on the outer side and inner side. The coating can be hydrophobic or hydrophilic depending on requirements.

In accordance with a further aspect of the invention, an insertion device for inserting a medical implant into a human or animal body is proposed and is designed to cooperate with an insertion element according to the invention.

Due to the two-dimensional, flat profile of the insertion element, the insertability into the human or animal body can be improved, and therefore complications can be reduced. The miniature insertion element in particular facilitates the research of new implants in animal experiments.

### DESCRIPTION OF THE DRAWINGS

The invention is explained in greater detail hereinafter by way of example on the basis of exemplary embodiments illustrated in drawings, in which:

FIG. 1 shows a schematic view of an insertion element in accordance with one embodiment of the invention in a first, unexpanded state in quasi two-dimensional form;

FIG. 2 shows a schematic view of the insertion element from FIG. 1 during the transition from the first state into a second, expanded state;

FIG. 3 shows a schematic view of the insertion element from FIG. 1 in the second, expanded state with the insertion device threaded through;

FIG. 4 shows a schematic view of an insertion element in accordance with a further embodiment of the invention in a first, unexpanded state in quasi two-dimensional form with a long, drawn-out tip at the distal end of the insertion element;

FIG. 5 shows a schematic view of an insertion element in accordance with a further embodiment of the invention in a first, unexpanded state in quasi two-dimensional form with a seam for removing the insertion element from the insertion device; and

FIG. 6 shows a schematic view of the insertion element according to FIG. 5 with an opened seam.

### DETAILED DESCRIPTION

In the figures, functionally like or similarly acting elements are denoted in each case by like reference signs. The figures are schematic illustrations of the invention. They do not show specific parameters of the invention. The figures also merely reproduce typical embodiments of the invention and are not intended to limit the invention to the embodiments illustrated.

FIGS. 1 to 3 show views of an insertion element 100 in accordance with one embodiment of the invention in a first, unexpanded state in quasi two-dimensional form (FIG. 1), during transition from the first state into a second, expanded state (FIG. 2), and an arrangement 200 of insertion element 100 in the second, expanded state and with the insertion device 220 threaded through the insertion element 100 (FIG. 3).

The insertion element 100 is designed to cooperate with an insertion device 220 for inserting a medical implant into a human or animal body and forms an arrangement 200 together with the insertion device 220.

The insertion element has a sleeve 10 with a distal end 40 and a proximal end 30 opposite the distal end 40. Here, the sleeve 10 in this embodiment has an expandable region 10a. This serves in the intended use as a sheath for the insertion device 220, which can be guided through the insertion element 100 through an opening 32 at the proximal end 30 and an opening 42 at the distal end 40. In this embodiment, the expandable region 10a forms the sleeve 10.

The clear cross-section of the opening 32 at the proximal end 30 is considerably smaller in the first state than in the second state. In the first state, the sleeve 10 or the expandable region 10a is flat and quasi two-dimensional. The sleeve 10 has an arrowhead-like contour in the first state, with the arrowhead at the distal end 40.

A fold 20 is formed along a longitudinal extension L of the sleeve 10, wherein a visible upper side 12 is formed from a first wing 12a on one side of the fold 20 and from a second wing 12b on the other side of the fold 20. An opposed rear side 14 (not visible in the figure) and also sides 16, 18 that can be folded between the front side 12 and the rear side 14 are formed comparably. The insertion element 100, or the sleeve 10, is opened by curving outwardly the edges of the wings 12a, 12b of the front side 12 and of the rear side and by lifting all folds 20 from the center line of the sleeve 10 so that, as the expanded state, the sleeve 10 forms a cone with a square cross section.

In the expanded state (FIG. 3), the side wings (of which the side wings 12a, 12b, 14a, 14b can be seen) are folded out and form a body that is symmetrical about a center axis and through which the insertion device 220 with an implant 222 can be passed.

The insertable region of the sleeve 10 is used as a sheath for the insertion device 220 with the medical implant 222, through which the insertion device is inserted into the human or animal body. The wall thickness of the sleeve 10 is advantageously very low, in particular considerably less than 500 μm thick, at most 100 μm thick, preferably at most 70 μm thick, and more preferably at most 50 μm thick. The sleeve 10 can be formed from a metal film or a polymer film and, where necessary, may have a friction-reducing coating.

The insertion device 220 is not illustrated in detail and may also comprise an inner shaft, on which the implant 222 is supported, as well as an outer shaft, which surrounds the inner shaft with the implant 222.

FIG. 4 shows a view of an insertion element 100 in accordance with a further embodiment of the invention in a first, unexpanded state in quasi two-dimensional form with a long, drawn-out tip 10b at the distal end 40 of the insertion element 100 and the unexpandable region 10a of said insertion element. The tip 10b allows an improved guidance of particularly thin insertion devices 220. Here, only the expandable region 10a of the sleeve 10 is expediently expandable, even in this example with an arrowhead-like contour, whereas the diameter of the long, drawn-out tip 10b does not necessarily change between the first state and the second state. The sleeve 10 can be manufactured from a single film, which is folded at one end and forms the expandable region 10a and, at the other end, the tip 10b.

FIGS. 5 and 6 shows a further embodiment of the insertion element 100, wherein the insertion element 100 has a seam 22 along a longitudinal extension L, which facilitates the removal of the insertion element 100 from the insertion device 220. Here, FIG. 6 shows how the seam 22 is opened from the proximal end 30 in order to peel the insertion element 100 from the insertion device 220. Here, the seam 22 forms a breaking seam in the sleeve 10.

To insert a catheter (insertion device 220) into the human or animal body, it is slid before-hand, together with the implant 220, through the insertion element 100 serving as a sheath. For insertion of the catheter, the flexible sheath is first inserted, for example into a blood vessel, and the catheter can slide through said sheath into the blood vessel. By opening the seam 22 (where provided), the insertion element 100 can be removed without difficulty from the insertion device 220 as required.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. An insertion element, which is designed to cooperate with an insertion device for inserting a medical implant into a human or animal body, characterized by a sleeve with a distal end and a proximal end opposite the distal end, the sleeve also having at least one expandable region, which can be transferred from a first state of small clear cross section at least at the proximal end into a second state of larger clear cross section at least at the proximal end, wherein the expandable region of the sleeve is flatter in the first state than in the second state.

2. The insertion element as claimed in claim 1, characterized in that the expandable region of the sleeve can be folded out and/or opened and/or fanned out from the first state into the second state.

3. The insertion element as claimed in claim 1, characterized in that the expandable region of the sleeve has an arrowhead-like contour in the first state.

4. The insertion element as claimed in claim 1, characterized in that the sleeve has a long, drawn-out tip at the distal end.

5. The insertion element as claimed in claim 1, characterized in that the sleeve has a seam along the longitudinal extension (L), wherein, when opening the seam, the interior at least of the expandable region can be exposed.

6. The insertion element as claimed in claim 1, characterized in that the sleeve, in the insertable region, which is provided as a sheath for the medical implant, has a wall thickness of at most 100 μm, preferably at most 70 μm, and more preferably at most 50 μm.

7. The insertion element as claimed in claim 1, characterized in that the sleeve is formed, at least in some regions, from a metal film having a wall thickness selected from the group consisting of at most 100 μm, at most 70 μm, and at most 50 μm.

8. The insertion element as claimed in claim 1, characterized in that the sleeve is formed, at least in some regions, from a polymer film having a wall thickness selected from the group consisting of at most 100 μm, at most 70 μm, and at most 50 μm.

9. The insertion element as claimed in claim 1, characterized in that the sleeve to has a friction-reducing coating.

10. An insertion device for inserting a medical implant into a human or animal body, the insertion device being designed to cooperate with an insertion element as claimed in claim 1.
